(19) United States
(12) Patent Application Publication
(10) Pub. No.: US 2008/0029706 A1
(43) Pub. Date: Feb. 7, 2008

(54) Particle therapy device

(76) Inventors: Werner Kaiser, Erlangen (DE); Hans Karcher, Karben (DE); Eike Rietzel, Darmstadt (DE); Ulrich Weis, Darmstadt (DE)

Correspondence Address: BRINKS HOFER GILSON & LIONE, P.O. BOX 10395, CHICAGO, IL, 60610, US

(21) Appl. No.: 11/809,645

(22) Filed: Jun. 1, 2007

(30) Foreign Application Priority Data
Jun. 6, 2006 (DE) DE 102006026212.3

(51) Int. Cl. A61B 6/00

(52) U.S. Cl. 250/363.02

(57) ABSTRACT

A particle therapy device is provided. The particle therapy device includes a gantry, which is rotatable about an axial axis of rotation and surrounds a treatment chamber with a floor that has at least one movable plate, a treatment table being positionable inside the treatment chamber. To make it possible for a patient, supported on the treatment table, to be irradiated from below as well, the plate is movable horizontally out of the treatment chamber in a transverse direction that extends perpendicular to the axial direction.

The present patent document claims the benefit of the filing date of DE 10 2006 026 212.3 filed Jun. 6, 2006, which is hereby incorporated by reference.

### BACKGROUND

The present embodiments relate to particle therapy equipment.

During a particle therapy treatment, especially for cancers, a particle beam is generated in a suitable accelerator. The particle beam, for example, comprises protons or heavy ions. The particle beam is guided in a radiation conduit and enters a treatment chamber via an exit window of the radiation conduit. Generally, only one stationary beam exit window is provided because of the complex course of the radiation. In some systems, however, a rotatable gantry with an exit window is provided. The gantry is constructed with a very large volume because of the complicated course of the radiation. The gantry surrounds an approximately cylindrical treatment chamber. A treatment table is moved into the cylindrical treatment chamber. For precise treatment, the patient's tissue to be irradiated must be positioned in the isocenter of the system.

A radiation unit generally includes at least one beam detector and passive beam elements disposed immediately in front of the exit window. To enable irradiating the patient from below, the gantry is ideally rotatable around the patient by 360°. However, a problem arises when the radiation unit is rotatable in the region below the patient. Accordingly, the floor of the treatment chamber opens, to allow the radiation unit to pass through the floor elements. However, at the same time, a floor in the treatment chamber is required. The floor is required so that the patient is accessible, maintenance work can be done, and so that there is no risk of falling for the equipment operators.

International Patent Disclosure WO 2004/026401 A1 discloses a radiation treatment chamber, which is a half-open room-sized space. The floor of this space is fixedly installed, except for an approximately 50 cm wide slit for the guidance of a radiation unit. The slit is covered with a covering. The gantry is rotatable by only 180°.

### SUMMARY

The present embodiments may obviate one or more of the limitations or drawbacks inherent in the related art. For example, in one embodiment, a particle therapy equipment includes a simple, compact structure, with which the patient can also be irradiated from below.

In one embodiment, a particle therapy device includes a gantry, which is rotatable about an axial axis of rotation and surrounds a treatment chamber with a floor that has at least one movable plate. A treatment table is positionable inside the treatment chamber. The movable plate is movable horizontally out of the treatment chamber in a transverse direction that extends perpendicular to the axial direction. The plate may be moved to enable a radiation unit and/or a counterweight to pass through it.

One or more plates may be moved in a transverse direction horizontally out of the treatment chamber. The plates that have been moved away do not impede the rotation of a radiation unit of the gantry or of a counterweight diametrically opposite the radiation unit. Moving the plates horizontally is technologically easy to achieve because a simple load-bearing construction is sufficient. The engineering effort and expense for moving the plates is slight.

In one embodiment, the floor includes a plurality of plates. Only those plates that are at risk of collision with the radiation unit of the gantry, for example, in a peripheral region of the floor, are ever removed. The floor in the treatment chamber remains accessible to people over a large area. The floor may be moved such that moving one or more plates out has no negative affects on the rigidity of the remaining floor.

In an alternate embodiment, the floor includes only a single plate. The single plate may be moved partially or completely out of the treatment chamber. The movement of the single plate depends on the position of the radiation unit or of the counterweight. The plate may be displaced laterally with a simple, linear motion. The openings in the floor, which are created by displacement of the plate, are at least partly covered in many cases by the radiation unit and the counterweight. The movable plate may be supported in such a way that it can be displaced horizontally in opposite directions. If there is a risk of collision with the plate from one side, then the plate is moved accordingly in the direction of the opposite side in such a way that the collision is avoided over an area of the floor that remains as large as possible.

The jacket face of the approximately cylindrical treatment chamber may be formed by a fixed wall of the room on which the components of the gantry are supported, for example, the radiation unit and the counterweight. Receptacles for the at least one plate may be provided in the axially extending solid wall that laterally defines the treatment chamber. These receptacles may be embodied on both sides of the treatment chamber. The one or more plates have great freedom of motion. The receptacles may be located approximately at the level of the floor and are adapted to the size and shape of the plates. The plates that form the floor can be simultaneously supported outside the treatment chamber.

In one embodiment, the plate is supported movably on a fixed floor that on its face end adjoins the treatment chamber. The fixed floor is a firm foundation. The load-bearing construction and the movement mechanism of the plates are mounted on the fixed floor. The fixed floor represents a construction that is independent from the treatment chamber and from the gantry and is not connected to them. A back wall of the treatment chamber, which is diametrically opposite the face end of the treatment chamber, may rotate with it but the floor remains still.

In one embodiment, on a face end of the fixed floor oriented toward the treatment chamber, a guide element extending in the transverse direction supports and moves the plate. The guide element is long enough that the at least one plate can be moved all the way outside the treatment chamber. The guide element is designed in such a way that the stability of the fixed floor is not impaired. The guide element is disposed on the face end of the fixed floor. On this side of the fixed floor, the load-bearing and movement mechanism of the plates is protected against unintentional damage, and proper operation is assured.

A central base plate and two side plates may be provided. The width of each of the side plates amounts to approximately 15% to 25% of the total width of the floor. The side plates may also have a limited load-bearing capacity. The side plates may serve to protect against falling. If the radiation unit and the counterweight come closer to the floor without having to be moved into it and back out again, then only the side plates are pushed out of the way, while the base plate remains unmoved and offers adequate load-bearing capacity.

In one embodiment, the side plates are pivotably supported. The space required in the transverse direction is reduced, since only the base plate has to be moved horizontally outward.

In one embodiment, the side plates are supported in such a way that they are pivotable both upward and downward relative to the floor. This takes into account the direction from which the radiation unit and the counterweight approach, and the side plates are pivoted accordingly, thus optimizing the course of motion of the plates. If the radiation unit, which is movable clockwise, approaches the side plate on the right on the face end from above, for example, then the side plate is correspondingly hinged downward. At the same time, the side plate on the left is pivoted upward, to create space for the counterweight that is diametrically opposite the radiation unit.

The pivot shaft of the side plates may be disposed above the base plate. Because the pivot shafts are spaced apart somewhat from the receptacles for the base plate, enough space is available to embody the receptacles.

In one embodiment, the at least one movable plate has rollers for support on the guide element. The plate can be moved back and forth easily using the rollers. The forces of friction are minimal. The path of motion of the rollers is predetermined by the guide element. Since the plate is supported on only one end, high torque acts on it. The axles on which the rollers are supported and the rollers themselves should be capable of withstanding the load on the plate from its own weight and from further loads. The axles and rollers are solid. The rollers can bear the weight of the plate as well as the weight of at least one person standing (resting) on the plate. A large number of wheels and axles may be provided to distribute the load among the individual axles.

The floor may have a plurality of horizontally movable plates. The horizontally movable plates are positionable one above the other laterally outside the treatment chamber. The plates that are moved out first may be put in a higher or lower parking position, and thus the following plates can be parked below and above them. Because of this layered arrangement of the plates that have been moved away, the dimensions of the receptacles in the transverse direction may be small. Different guide elements may be provided for the different plates. These guide elements may be offset slightly from one another in height, or in such a way that no substantial gaps between the plates of the floor are created. For example, if the floor comprises one base plate and two side plates, then a longer central guide element for the base plate may be provided, and two lateral guide elements for the side plates may be offset from it somewhat in height.

In one embodiment, the guide element, in the region laterally outside the treatment chamber, branches into two split arms extending one above the other. One of the split arms extends horizontally, as an extension of the guide element. The plates, and in particular the somewhat wider plates, if the plates are designed with different widths, can easily be moved horizontally back and forth. The plates that have been moved away are likewise parked one above the other, thus occupying little space in the transverse direction away from the treatment chamber.

The at least one plate may be bent at an angle in the shape of an L toward the face end of the fixed floor. A plurality of guide elements may be provided in the radial direction. The radially downward-extending part of the plate, bent at an angle, is shaped in the order of an angle bracket, which is braced at a plurality of points on the fixed floor via the guide elements. The angle bracket extends radially downward only far enough that there is no risk of collision with the counterweight.

In one embodiment, the guide element is a linear guide, for example, a guide rail or a guide groove. The linear guide allows a translational motion of the plate along a defined path of motion. The linear guide allows the plates to be pushed back and forth.

Imaging systems, such as an X-ray tube and a diametrically opposed X-ray detector, may be integrated with this kind of particle therapy equipment. The X-ray detector and the X-ray tube may be mounted on the radiation unit and the counterweight, respectively. Because the imaging system rotates with the gantry, and by a gantry revolution, a volumetric image of diseased tissue of the patient can easily be generated. The plate may have a radiotransparent region, for example, comprising a carbon fiber reinforced plastic. The radiotransparent region may have the shape, for example, of strip extending in the transverse direction.

A robot secured to the fixed floor may be provided for positioning the treatment table inside the treatment chamber. The treatment table may be moved into the treatment chamber, which is surrounded by the gantry, and held there without the treatment table being in contact with the floor. Accordingly, great stability of the treatment table may be assured because its position in the treatment chamber is independent of the particular position and disposition of the plates.

### BRIEF DESCRIPTION OF THE DRAWINGS

FIG. 1 is a front view of one embodiment of a particle therapy device having a floor that includes a displaceable base plate and two pivotable side plates;

FIG. 2 is a section taken along the line B-B of FIG. 1;

FIG. 3 is a front view of one embodiment of a particle therapy device having a floor that includes a plurality of displaceable plates;

FIG. 4 illustrates a support of a plate; and

FIG. 5 is a front view of one embodiment of a particle therapy device having a floor that includes only one plate.

### DETAILED DESCRIPTION

The same reference numerals generally have the same meaning throughout the drawings.

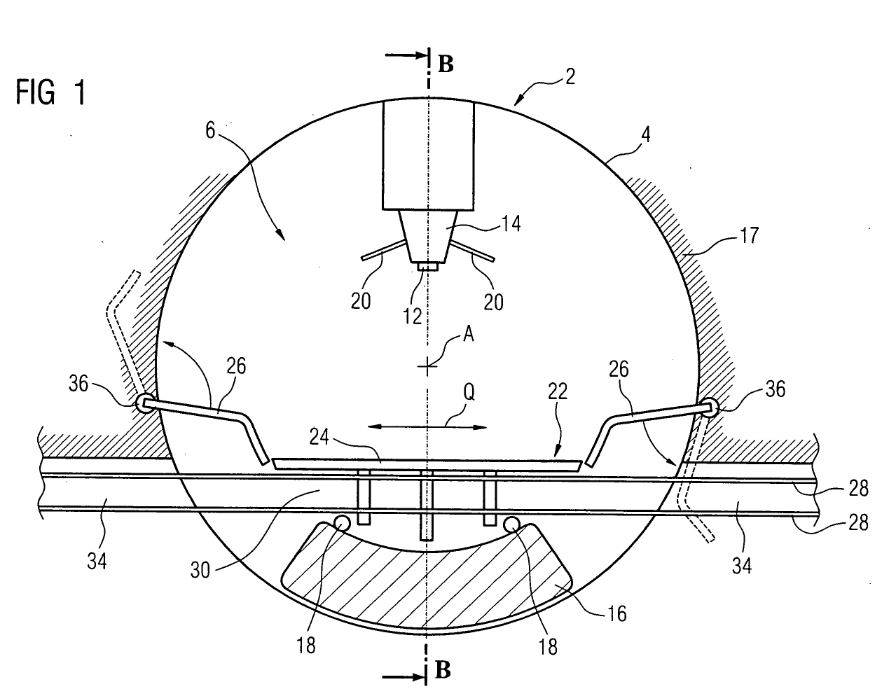

In one embodiment, as shown in FIG. 1, a particle therapy device 2 includes a gantry 4 that is rotatable about an axis of rotation A (represented by a dot) extending in the axial direction. The gantry 4 may be rotatable by 360°. The gantry 4 surrounds an approximately cylindrical treatment chamber 6. In a radiation conduit of the gantry 4, a particle beam, such as a heavy ion beam or a proton beam, is generated for treating a patient 10 lying on a treatment table 8. The particle beam enters the treatment chamber 6 via an exit window 12 of a radiation unit 14. The gantry 4 may include a counterweight 16, which is disposed diametrically opposite the radiation unit 14 and rotates accordingly with it. The gantry 4 is supported rotatably on a fixed wall 17 that laterally defines the treatment chamber 6 and extends axially.

An imaging system may be used for imaging diseased tissue of the patient. The imaging system may be integrated with the gantry 4. The imaging system may include two X-ray tubes 18 disposed on the counterweight, and two X-ray detectors 20 mounted on the radiation unit 14. The X-ray beams emitted by the X-ray tubes 18 intersect an isocenter of the gantry 4. As shown in FIG. 1, the isocenter matches the point A in which the diseased tissue of the patient 10 is positioned during the particle therapy. The information obtained as a result of the X-rays, after being evaluated, furnishes 3D views of the diseased tissue.

In one embodiment, the treatment chamber 6 includes a floor 22, which comprises one central base plate 24 and two side plates 26. The base plate 24, in this exemplary embodiment, is approximately the same width as the side plates 26. The base plate 24 has a load-bearing function and has a thickness that lends it adequate rigidity when loaded by the weight of at least one person. The base plate 24 is designed such that it can be moved horizontally out of the treatment chamber 6 in a transverse direction Q that is indicated by a double arrow. Alternatively, two or more plates may be moved out of the treatment chamber 6 to the left and right in the transverse direction Q.

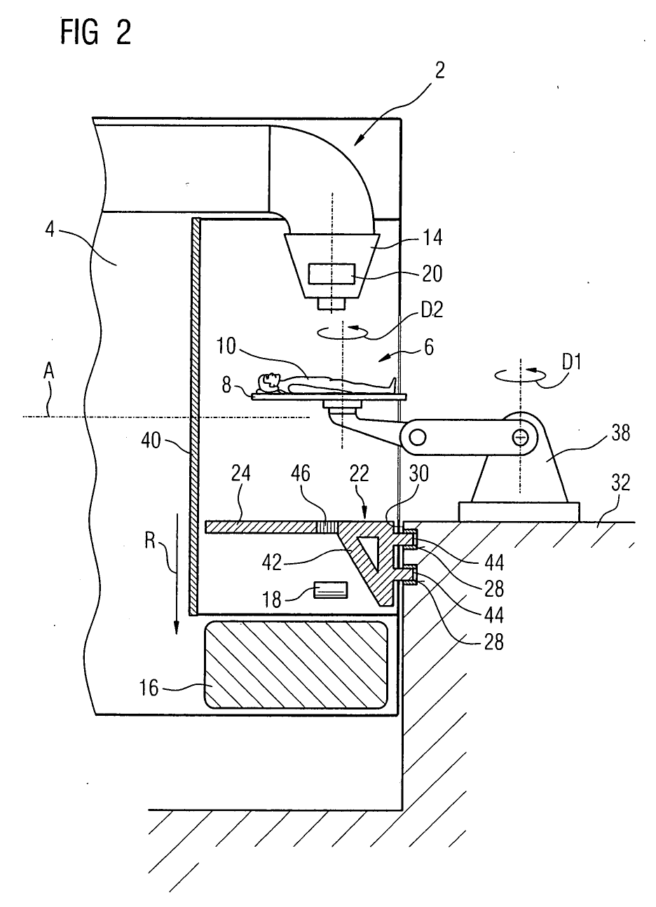

Two guide rails 28 may extend in the transverse direction Q to enable moving the base plate 24 horizontally. As shown in FIG. 2, the guide rails 28 are mounted on a face end 30, toward the treatment chamber 6, of a fixed floor 32. This fixed floor 32 adjoins the treatment chamber 6 and, for example, is at the level of the base plate 24.

Receptacles 34 are embodied in the fixed wall 17 on both sides of the treatment chamber 6. The base plate 24 that has been moved out of the treatment chamber is accommodated in the receptacles 34. The receptacles 34 are located approximately at the level of the base plate 24 and of the fixed floor 32. The receptacles 34 are adapted to the size and shape of the base plate 24, so that the base plate 24 can be moved all the way out of the treatment chamber 6.

Two pivot shafts 36 may be provided on both sides of the treatment chamber 6, approximately above the base plate 24. The two pivot shafts 36 support the side plates 26. The side plates 26 may be used solely to protect against falling and cannot be walked on or driven on. The side plates 26 may be disposed at a somewhat higher level than the base plate 24, and have inclined surfaces. The side plates 26 can be pivoted both upward and downward, which is shown in the drawing with arrows and dashed lines. In their pivoted position, the side plates 26 are located all the way outside the treatment chamber 6, so that they do not restrict the motion of the radiation unit 14 and of the counterweight 16. The fixed wall 17 may include recesses so that the side plates 26 can be pivoted all the way outside the treatment chamber 6. These recesses may be widened portions of the receptacles 34.

The side plates 26 may have the function of pinch protection on approaching the radiation unit 14. The side plates 26 have a sensor that detects body parts and objects that are caught and communicates the fact, so that the motion of the gantry 4 is brought to a standstill as needed.

The guide rails 28 and the pivot shafts 36 are a construction that is independent of the gantry 4. The base plate 24 and the side plates 26 may be moved in a way that is decoupled from the gantry 4. The base plate 24 and the side plates 26 motion are coordinated with the motion of the gantry 4. A control unit may move the base plate 24 and the side plates 26 away as a function of the position of the radiation unit 14 and of the counterweight 16.

In one embodiment, as shown in FIG. 2, the treatment table 8 is positioned in the treatment chamber 6 and is movable using a patient handling system. The patient handling system may comprise a triggered robot 38. The treatment table 8 has no contact with the floor 22. The robot 38 is mounted on the fixed floor 32 outside the treatment chamber 6. The robot 38 is a multiaxial industrial robot with a multi-part mechanism. The treatment table 8 may be moved translationally in both the horizontal and the vertical directions using the robot 38. Both rotation of the robot 24 about an axis of rotation D1 perpendicular to the fixed floor 32 and rotation of the treatment table 8 about a further axis of rotation D2 are possible. The diseased tissue of the patient 10 may be positioned in the isocenter of the gantry 4 using the translational and rotary motions of the treatment table 8. During the positioning and irradiation of the patient 10, the treatment table 8 remains in a horizontal position, so the patient 10 lies stably on the treatment table 8.

As shown in FIG. 2, the fixed floor 32 adjoins the treatment chamber 6 and faces toward it with its face end 30. The term “treatment chamber” in this exemplary embodiment is understood as the cylindrical space surrounded by the gantry 4. This space is defined laterally by the fixed wall 17 and at the back by a back wall 40 and being partly open on the front or face end. Since the gantry 4 and the floor 22 form separate units whose motions are decoupled from one another, and the plates 24, 26 are not supported on the back wall 40. The back wall 40 may rotate with the gantry 4. The back wall 40 may be simple cladding, without any mechanical load-bearing capacity.

The treatment chamber 6 may be subdivided by the floor 22 into an upper part and a lower part. The upper part is open to the front, so that the treatment table 8 can be moved into it. In the lower part, in the region where there is no risk of a collision with the counterweight 16, there is essentially a motion mechanism of the plate 24. The plate 24 includes the two guide rails 28 disposed one above the other in a radial direction R, and a plurality of angle brackets 42 that are coupled to the guide rails 28.

As shown in FIG. 2, the base plate 24 is bent at an angle toward the face end 30 of the fixed floor 32. The base plate 24 may form an angle bracket 42. Sliding or rolling elements 44 of the plate 24 are moved into the two guide rails 28 and make simple displacement of the plates 24 possible with minimized friction. The sliding or rolling elements 44 may include, for example, rollers, which roll in the path of motion defined by the guide rails 28.

The base plate 24 may include a radiotransparent region 46, for example, comprising a carbon fiber reinforced plastic. The diagnostic imaging of the patient 10 is possible with the aid of the X-ray tubes 18 and the detectors 20.

During therapy, the patient 10 is first immobilized on the treatment table 8. The treatment table 8 is moved into the treatment chamber 6 of the gantry 4 via the robot 38 and positioned in such a way that the diseased tissue of the patient 10 is located in the isocenter of the gantry 4. To set an angle for the irradiation, the gantry 4 may be rotated clockwise about its axis of rotation R. When the radiation unit 14 rotates, the back wall 40 of the treatment chamber 6 rotates with it. There is no relative motion of the radiation unit 14 relative to the back wall 40.

Because of the relatively large volume of the radiation unit 14, there is a risk of collision between it and the floor 22, both in the peripheral region of the floor 22 and under the floor 22. A collision may be avoided by the control unit. The control unit moves the side plates 26 and optionally the base plate 24 part of the way or all of the way into the receptacles 34 in the fixed wall 17. The control unit moves the side plates 26 and optionally the base plate 24 as a function of the position of the radiation unit 14. The control unit may open at least part of the floor 22. The resultant opening in the floor 22 is partly covered by the radiation unit 14, so that the risk of falling, for the workers or for objects, is reduced. The floor 22 is triggered in such a way that with a stationary gantry 4, the access to the patient 10 is assured via at least a portion of the plate 24. The floor 22 is virtually gap-free, so that it can be driven upon with equipment, for example, an anesthesia trolley, patient shuttle, IV stands, and service and QA equipment.

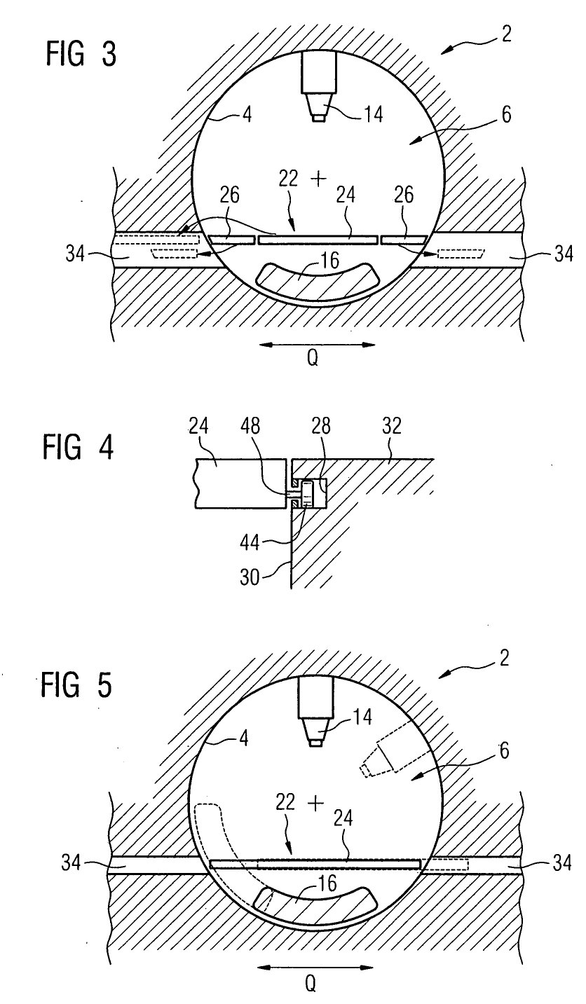

In one embodiment, as shown in FIG. 3, the floor 22 comprises one larger base plate 24 and two smaller side plates 26. The plates 24, 26 are movable horizontally out of the treatment chamber 6 of the gantry 4 via a guide rail. In this exemplary embodiment, the side plates 26, like the base plate 24, are embodied as load-bearing elements of the floor 22.

The fixed wall 17 may include receptacles 34 on both sides of the treatment chamber 6. The receptacles 34 are disposed high enough that the plates 24, 26 can be supported one above the other in a parking position. For example, as the arrows indicate in FIG. 3, if there is a risk of collision, first the side plates 26 are moved into the receptacles 34 and parked in a lower position. The base plate 24 may be also moved out of the treatment chamber 6 and parked in one of the receptacles 34, above the side plate 26 located there. The receptacles 34 have a width in the transverse direction Q that is at least equivalent to the width of the base plate 24, so that the base plate 24 may be moved all the way into the receptacles 34.

To achieve positioning of the plates 24, 26 one above the other, the guide rail 28, in the region laterally outside the treatment chamber 6, may branch into two split arms extending one above the other.

In one embodiment, as shown in FIG. 4, the plates 24, 26 may be supported on the fixed floor 32. Each of the plates 24, 26, on a side toward the guide rail 28, includes a number of axles 48. A sliding or rolling element 44 on the order of a roller is supported on the number of axles 48. The axles 48 and the rollers 44 hold the weight of the plate 24, 26 as well as at least the weight of one person standing on the plate 24, 26.

In one embodiment, as shown in FIG. 5, the floor 22 comprises only one plate 24, which is movable in the transverse direction Q. If a collision, for example, with a counterweight 16, threatens, then the plate 24 is shifted slightly to the right, as indicated by dashed lines. The plate 24 is shifted so that enough space is created on the left side for the counterweight 16 that is rotating clockwise. If upon further rotation of the gantry 4 the counterweight 16 has come all the way up out of the floor 22 on the left, while there is a risk of collision with the radiation unit 14 on the right, the plate 24 is shifted horizontally, for example, to the left, far enough that the radiation unit 14 can plunge underneath the floor 22 without problems. The gaps created in the floor 22 are always covered by the counterweight 16 or the radiation unit 14. At every rotational angle of the gantry 4, there is at least some floor 22 present in the treatment chamber 6.

In one embodiment, the structure and the type of movement of the at least one plate 24 horizontally out of the treatment chamber 6 allow irradiation of the patient 10 from below and accessibility to the patient 10 is assured at all times.

Various embodiments described herein can be used alone or in combination with one another. The forgoing detailed description has described only a few of the many possible implementations of the present invention. For this reason, this detailed description is intended by way of illustration, and not by way of limitation. It is only the following claims, including all equivalents that are intended to define the scope of this invention.

1. A particle therapy device, including:
a gantry that is rotatable about an axis of rotation;
a treatment chamber with a floor having at least one movable plate, the gantry surrounding the treatment chamber; and
a treatment table positionable inside the treatment chamber,
wherein the at least one movable plate is movable horizontally out of the treatment chamber in a transverse direction that extends perpendicular to the axial direction.

2. The particle therapy device as defined by claim 1, comprising receptacles for the at least one movable plate, wherein the receptacle are provided in an axially extending solid wall that laterally defines the treatment chamber.

3. The particle therapy device as defined by claim 1, wherein the at least one movable plate is supported movably on a fixed floor.

4. The particle therapy device as defined by claim 3, wherein on a face end of the fixed floor oriented toward the treatment chamber, a guide element, which extends in the transverse direction, supports and moves the at least one movable plate.

5. The particle therapy device as defined by claim 1, comprising a central base plate and two side plates.

6. The particle therapy device as defined by claim 5, wherein the side plates are pivotably supported.

7. The particle therapy device as defined by claim 6, wherein the side plates are pivotable both upward and downward relative to the floor.

8. The particle therapy device as defined by claim 7, wherein a pivot shaft of the side plates is disposed above the base plate.

9. The particle therapy device as defined by claim 8, wherein the at least one movable plate comprises rollers for support on the guide element.

10. The particle therapy device as defined by claim 1, wherein the floor comprises a plurality of horizontally movable plates.

11. The particle therapy device as defined by claim 4, wherein the guide element, in the region laterally outside the treatment chamber, branches into two split arms extending one above the other.

12. The particle therapy device as defined by claim 4, wherein the at least one plate is bent at an angle in the shape of an L toward the face end of the fixed floor; and that in the radial direction, a plurality of guide elements are provided.

13. The particle therapy device as defined by claim 4, wherein the guide element is a linear guide, in particular a guide rail or a guide groove.

14. The particle therapy device as defined by claim 1, wherein the at least one movable plate comprises a radiotransparent region.

15. The particle therapy device as defined by claim 1, comprising a robot is operable to position the treatment table.

16. The particle therapy device as defined by claim 3, wherein one end of the fixed floor adjoins the treatment chamber.

17. The particle therapy device as defined by claim 7, wherein a width of each of the side plates amounts to approximately 15% to 25% of the total width of the floor.

18. The particle therapy device as defined by claim 10, wherein the horizontally movable plates are positionable one above the other laterally outside the treatment chamber.

19. The particle therapy device as defined by claim 15, wherein the robot is secured to the fixed floor.
